(19) United States
(12) Patent Application Publication
(10) Pub. No.: US 2023/0270978 A1
(43) Pub. Date: Aug. 31, 2023

(54) Catheter Adapter with Stabilization Feature

(71) Applicant: Becton, Dickinson and Company, Franklin Lakes, NJ (US)

(72) Inventors: John M. Lackey, West Valley City, UT (US); Ralph L. Sonderegger, Farmington, UT (US); Nathan Aaron Mitchell, Murray, UT (US); Justin G. Hortin, Farmington, UT (US); Weston F. Harding, Lehi, UT (US)

(21) Appl. No.: 18/113,347

(22) Filed: Feb. 23, 2023

Related U.S. Application Data
(60) Provisional application No. 63/313,821, filed on Feb. 25, 2022.

(51) Int. Cl. A61M 25/02; A61M 25/00

(52) U.S. Cl. CPC A61M 25/02; A61M 25/0097; A61M 2205/0216

(57) ABSTRACT

A catheter adapter includes a body having a first end and a second end positioned opposite the first end, with the body including an inlet port positioned between the first end and the second end and defining a longitudinal axis extending between the first end and the second end of the body, a catheter configured to be inserted into a patient's vasculature, with the catheter positioned at the first end of the body, and a stabilizer including a first wing member and a second wing member, with a portion of the stabilizer extending beyond the inlet port of the body in a direction extending along the longitudinal axis from the first end of the body to the second end of the body.

### CROSS-REFERENCE TO RELATED APPLICATION

The present application claims priority to U.S. Provisional Application Ser. No. 63/313,821, entitled “Catheter Adapter with Stabilization Feature”, filed Feb. 25, 2022, the entire disclosure of which is hereby incorporated by reference in its entirety.

### BACKGROUND OF THE INVENTION

### Field of the Invention

The present disclosure relates to a catheter adapter with a stabilization feature.

### Description of Related Art

Catheters are frequently utilized to administer fluids into and out of the body. Patients in a variety of settings, including in hospitals and in home care, receive fluids, pharmaceuticals, and blood products via a vascular access device inserted into a patient's vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient's vein. The catheter length may vary from a few centimeters for peripheral access to many centimeters for central access. The catheter is commonly incorporated into a catheter adapter to aid in the ease of use, accessibility and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional infusion components for use with the catheter. For example, the second end of a catheter adapter may include a set of threads for attaching an intravenous line or for coupling a syringe to the catheter adapter thereby providing access to the patient's vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient's vein and provide an opening for insertion of the catheter.

During insertion into a patient, the needle and catheter generally approach the patient's vein at an angle of about 30° where the needle initially punctures the patient's epidermis and then continues into the vein. Once the needle and catheter tip enter the patient's vein, the needle and catheter are then repositioned so that the needle and catheter are brought into a position generally parallel with the patient's vein so that the needle and catheter may be inserted into the lumen of the patient's vein. When the catheter has been properly positioned within the patient's vein, the needle is removed from the lumen of the catheter and the catheter adapter is secured to the patient to prevent premature removal of the catheter.

The catheter adapter may be secured to the patient by fastening the catheter adapter to the patient's skin via tape, a securement device, and/or a securement dressing. When securing the catheter adapter to the patient's skin, the root region of the catheter immediately exiting the catheter adapter must arch to accommodate the catheter's transition from the generally parallel, secured orientation of the catheter adapter, to the insertion angle of the catheter.

Referring to FIG. 1, an extended section of catheter may be left between the patient and the catheter adapter to allow for transitional arching of the catheter. This exposed, archable length of catheter biases the catheter towards the patient's skin. The portion of the catheter extending from the catheter adapter may generally follow an S-shaped profile. The catheter, however, may kink slightly at the bends of the S-shaped profile, which can occlude the catheter and/or inhibit the passage of instruments through the catheter. For example, the S-shaped profile and/or kinks in the catheter can pose a challenge for line draw devices, which utilize the catheter and pass through the catheter to open up a fluid path for blood draw.

### SUMMARY OF THE INVENTION

In one aspect or embodiment, a catheter adapter includes a body having a first end and a second end positioned opposite the first end, with the body including an inlet port positioned between the first end and the second end and defining a longitudinal axis extending between the first end and the second end of the body, a catheter configured to be inserted into a patient's vasculature, with the catheter positioned at the first end of the body, and a stabilizer including a first wing member and a second wing member, with a portion of the stabilizer extending beyond the inlet port of the body in a direction extending along the longitudinal axis from the first end of the body to the second end of the body.

A bottom surface of the stabilizer may be configured to contact a skin surface of a patient, and where the bottom surface of the stabilizer defines a plane forming an angle greater than zero relative to the longitudinal axis of the body.

The stabilizer may be symmetric with respect to the longitudinal axis. The stabilizer may be asymmetric with respect to the longitudinal axis.

The first wing member may include a first end and a second end, with the first wing member defining a recessed portion adjacent to the inlet port of the body and the recessed portion of the first wing member extending from the second end of the first wing member toward the first end of the first wing member.

The first and second wing members may each define an opening positioned entirely within a perimeter of the first and second wing members.

The stabilizer may include a first extension portion extending from the first wing toward the second end of the body and a second extension portion extending from the second wing toward the second end of the body.

The stabilizer may include a clip configured to attach the stabilizer to the body. The clip may be positioned on a base portion, with the base portion forming a portion of the stabilizer.

The portion of the stabilizer extending beyond the inlet port of the body may include a hinged portion. The hinged portion may include a living hinge.

A bottom surface of the stabilizer may be configured to contact a skin surface of a patient, where the bottom surface of the stabilizer defines a recess configured to align with a vein of a patient when the stabilizer is in contact with a skin surface of a patient.

A bottom surface of the stabilizer may be configured to contact a skin surface of a patient, where the bottom surface of the stabilizer comprises a plurality of protrusions and/or a plurality of channels.

In a further aspect or embodiment, a vascular access device includes a catheter adapter of any of the aspects or embodiments discussed above and a needle hub assembly connected to the body of the catheter adapter.

The stabilizer may be spaced from the needle hub assembly.

In a further aspect or embodiment, a catheter adapter includes a body having a first end and a second end positioned opposite the first end, with the body including an inlet port positioned between the first end and the second end and defining a longitudinal axis extending between the first end and the second end of the body, a catheter configured to be inserted into a patient's vasculature, with the catheter positioned at the first end of the body, a first support member extending from the body, and a second support member extending from the body, with the first and second support members configured to engage a skin surface of a patient and space the body from the skin surface of the patient when the catheter is inserted into the patient.

The first and second support members may each include a support surface spaced from a bottom surface of the body via a support arm. The first support member may be positioned on an opposite side of the body from the second support member. The support surface of each of the first and second support members may include an elastomeric material.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a partial cross-sectional view of a conventional catheter adapter and catheter, showing the catheter inserted into a vein of a patient;

FIG. 2 is a perspective view of a vascular access device according to one aspect or embodiment of the present application;

FIG. 3 is a side view of a conventional catheter adapter;

FIG. 4 is a side view of a catheter adapter according to one aspect or embodiment of the present application;

FIG. 5 is a top view of a catheter adapter according to one aspect or embodiment of the present application;

FIG. 6 is a top view of a catheter adapter according to one aspect or embodiment of the present application;

FIG. 7 is a top view of a catheter adapter according to one aspect or embodiment of the present application;

FIG. 8 is a top view of a catheter adapter according to one aspect or embodiment of the present application;

FIG. 9 is a top view of a catheter adapter according to one aspect or embodiment of the present application;

FIG. 10 is a bottom view of a catheter adapter according to one aspect or embodiment of the present application;

FIG. 11 is a top view of a catheter adapter according to one aspect or embodiment of the present application;

FIG. 12 is a bottom view of a catheter adapter according to one aspect or embodiment of the present application;

FIG. 13 is a bottom view of a catheter adapter according to one aspect or embodiment of the present application;

FIG. 14 is a bottom view of a catheter adapter according to one aspect or embodiment of the present application;

FIG. 15 is a bottom view of a catheter adapter according to one aspect or embodiment of the present application;

FIG. 16 is a bottom view of a catheter adapter according to one aspect or embodiment of the present application;

FIG. 17 is a side view of a catheter adapter according to one aspect or embodiment of the present application;

FIG. 18 is a top view of a catheter adapter according to one aspect or embodiment of the present application;

FIG. 19 is a perspective view of a catheter adapter according to one aspect or embodiment of the present application;

FIG. 20 is a bottom view of a catheter adapter and a needle hub according to one aspect or embodiment of the present application;

FIG. 21 is a bottom view of a catheter adapter according to one aspect or embodiment of the present application;

FIG. 22 is a side view of a catheter adapter and a stabilizer assembly according to one aspect or embodiment of the present application;

FIG. 23 is a perspective view of the stabilizer assembly of FIG. 22;

FIG. 24 is a side view of a catheter adapter according to one aspect or embodiment of the present application;

FIG. 25 is a side view of the catheter adapter of FIG. 24, showing the catheter adapter in contact with a skin surface of a patient;

FIG. 26 is a side view of the catheter adapter of FIG. 24, showing a molding process for a stabilizer of the catheter adapter;

FIG. 27 is a bottom perspective view of a catheter adapter according to one aspect or embodiment of the present application; and

FIG. 28 is a perspective view of a catheter adapter according to one aspect or embodiment of the present application.

FIG. 29 is a perspective view of a catheter adapter according to one aspect or embodiment of the present application.

FIG. 30 is a perspective view of a catheter adapter according to one aspect or embodiment of the present application.

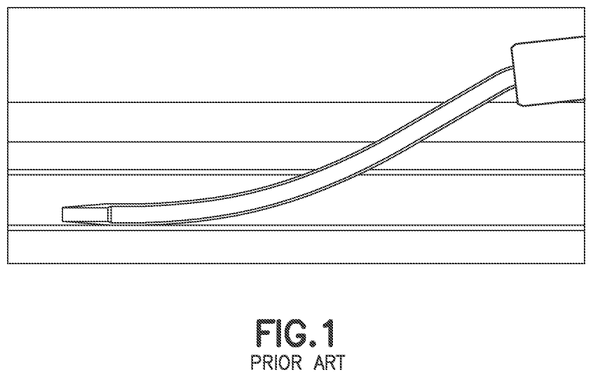

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

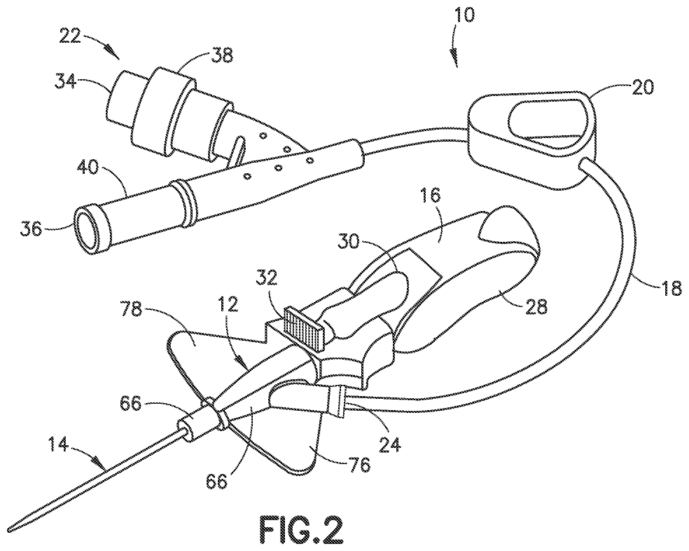

Referring to FIGS. 2 and 3, a vascular access device 10 according to one aspect or embodiment of the present application includes a catheter adapter 12 and a catheter 14. In certain aspects or embodiments, the vascular access device 10 further includes a needle hub assembly 16, an extension tube 18, a clamp 20, and a luer access device 22. The catheter adapter 12 includes an inlet port 24 that receives an end of the extension tube 18 for establishing fluid communication between an intravenous fluid source and the catheter adapter 12 or lumen of the catheter 14. In one aspect or embodiment, the needle hub assembly 16 is assembled with the catheter adapter 12 by inserting a needle into the lumen of the catheter 14. The needle hub assembly 16 is shown as including finger grips 28 positioned at the sides of the needle hub assembly 16 to facilitate various insertion techniques. In one aspect or embodiment, the needle hub assembly 16 includes a needle shield 30 configured to secure a tip of the needle within the shield after use. The needle shield 30 may be activated passively. The needle hub assembly 16 includes a push tab 32 to facilitate catheter advancement during insertion. The push tab 32 also allows for one-handed or two-handed advancement. The push tab 32 may be removed with the needle shield 30. The clamp 20 is positioned on the extension tubing 18 to prevent blood flow when necessary.

Referring to FIG. 2, the luer access device 22 of the vascular access device 10 includes a first luer access 34 and a second luer access 36 in fluid communication with the extension tube 18, a blood control split septum 38 associated with the first luer access 34, and an air vent 40 associated with the second luer access 36. The split septum 38 allows for a reduction in catheter-related bloodstream infection (CRBSI) while providing unrestricted flow and a straight fluid path and functions as a blood control septum 38. The air vent 40 allows air to escape from the system during insertion, providing continuous confirmation of vascular access while preventing leakage of blood from the system during insertion. In one aspect or embodiment, the extension tube 18 is connected near a patient port configured to be connected to a blood draw device, such as the PIVO™ blood draw device commercially available from Velano Vascular.

Referring to FIG. 3, a conventional catheter adapter 42 includes a stabilizing platform 44 positioned on a bottom surface of the conventional catheter adapter 42 that is made from an elastomeric material. The stabilizing platform 44 forms a “belly” that is in contact with a skin surface of a patient. During application of dressing to the conventional catheter adapter 42 and after placement of the catheter, the catheter can bend to prevent flow through the catheter and/or inhibit the passage of an instrument through the catheter.

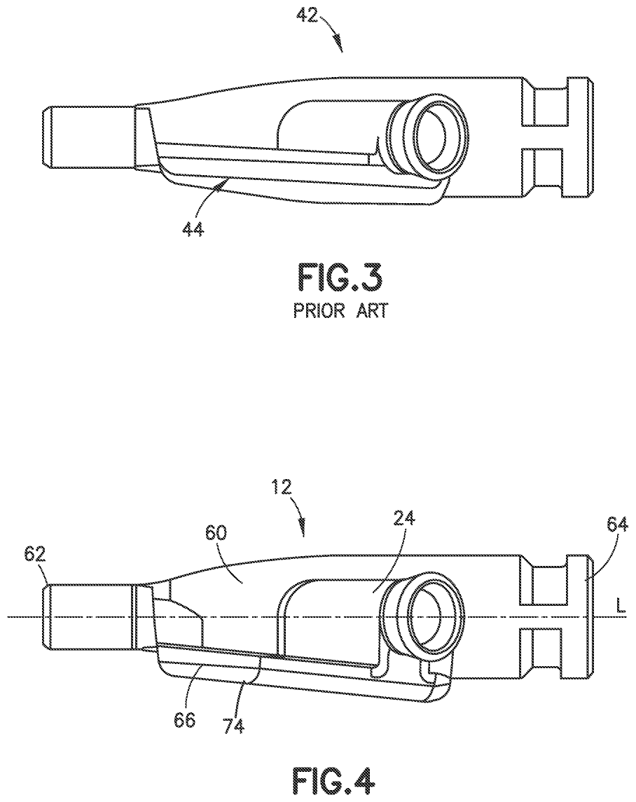

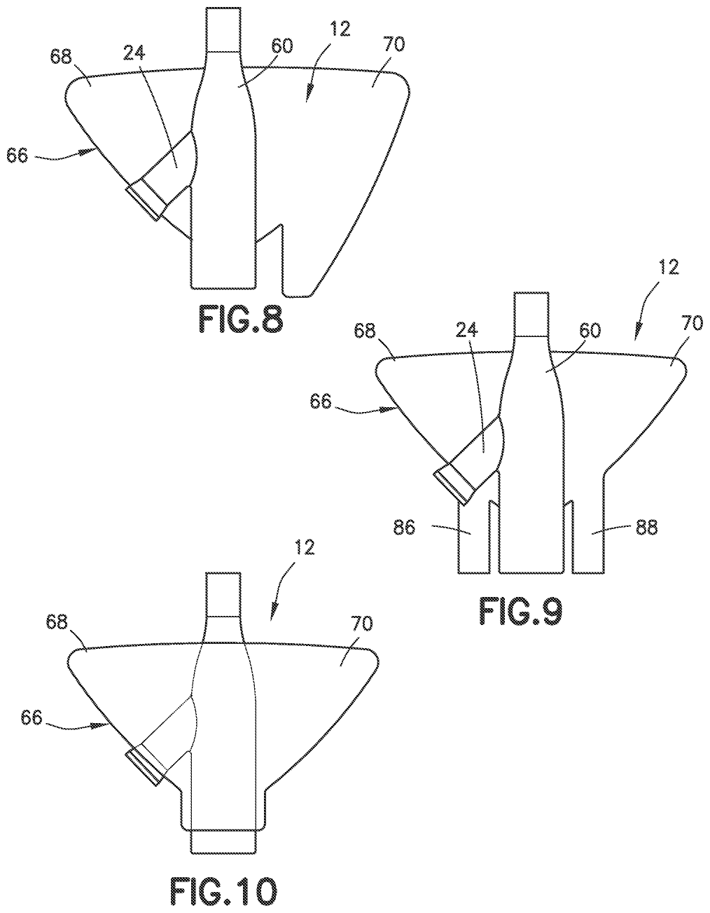

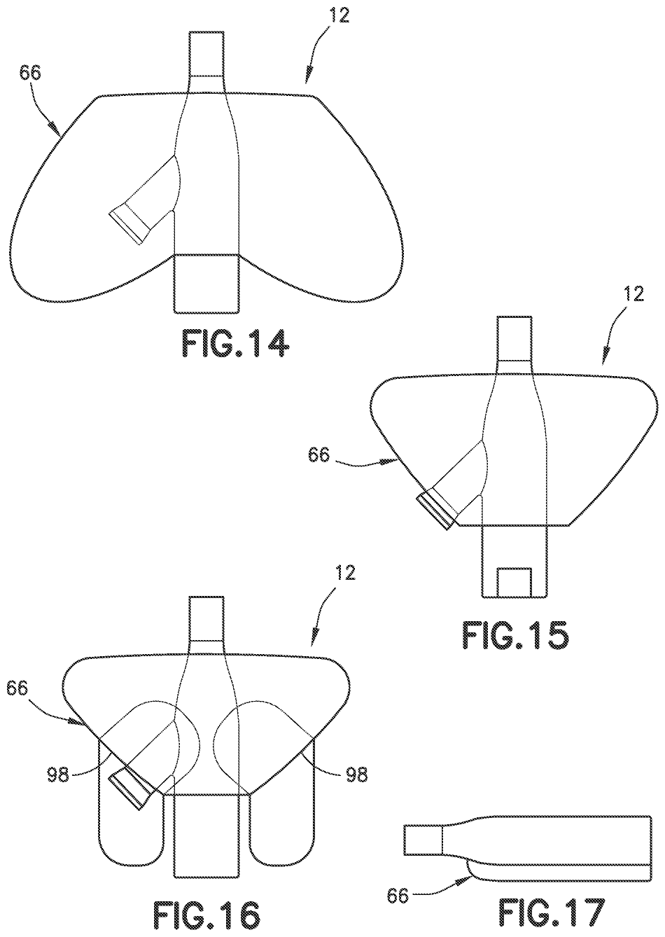

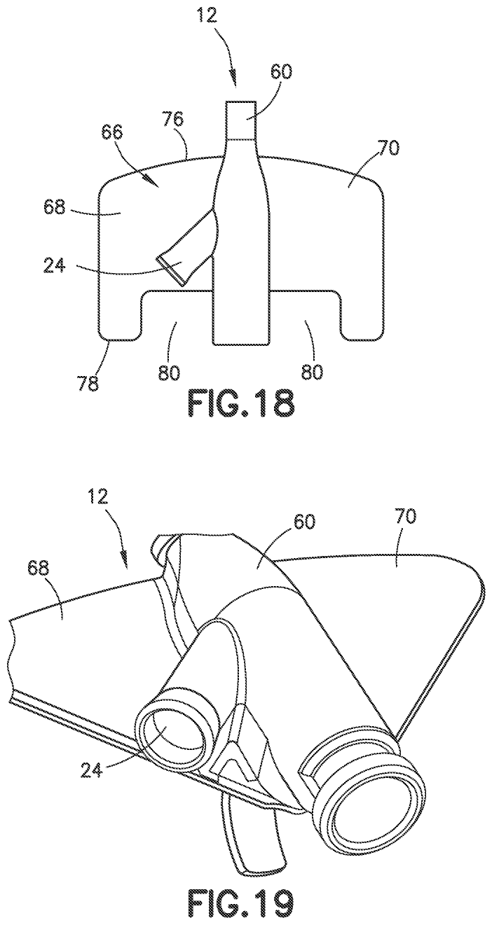

Referring to FIGS. 4-21, in one aspect or embodiment of the present application, the catheter adapter 12 of the vascular access device 10 includes a body 60 having a first end 62 and a second end 64 positioned opposite the first end 62. The inlet port 24 is positioned between the first end 62 and the second end 64. The body 60 defines a longitudinal axis L extending between the first end 62 and the second end 64 of the body 60. The catheter 14 is configured to be inserted into a patient's vasculature and is positioned at the first end 62 of the body 60. The catheter adapter 12 also includes a stabilizer 66 having a first wing member 68 and a second wing member 70. A portion of the stabilizer 66 extends beyond the inlet port 24 of the body 60 in a direction extending along the longitudinal axis L from the first end 62 of the body 60 to the second end 64 of the body 60. Providing more material of the stabilizer 66 toward the second end 64 of the body 60 stabilizes the catheter adapter 12 and prevents rocking of the catheter adapter 12, which can cause kinking or undesirable curvature of the catheter 14. The stabilizer 66 may be formed integrally and from the same material as the body 60. The stabilizer 66 may be formed from an elastomeric material. In one aspect or embodiment, the stabilizer 66 is formed utilizing a two-shot overmolding process.

Referring to FIG. 4, in one aspect or embodiment, a bottom surface 74 of the stabilizer 66, which is configured to contact a skin surface of a patient, defines a plane forming an angle greater than zero relative to the longitudinal axis L of the body 60. In one aspect or embodiment, the bottom surface 74 of the stabilizer 66 defines a plane forming an angle of 3-10 degrees relative to the longitudinal axis L of the body 60. The bottom surface 74 is configured to raise the second end 64 of the body 60 when engaged with a skin surface of a patient to reduce kinking or undesirable curvature of the catheter 14.

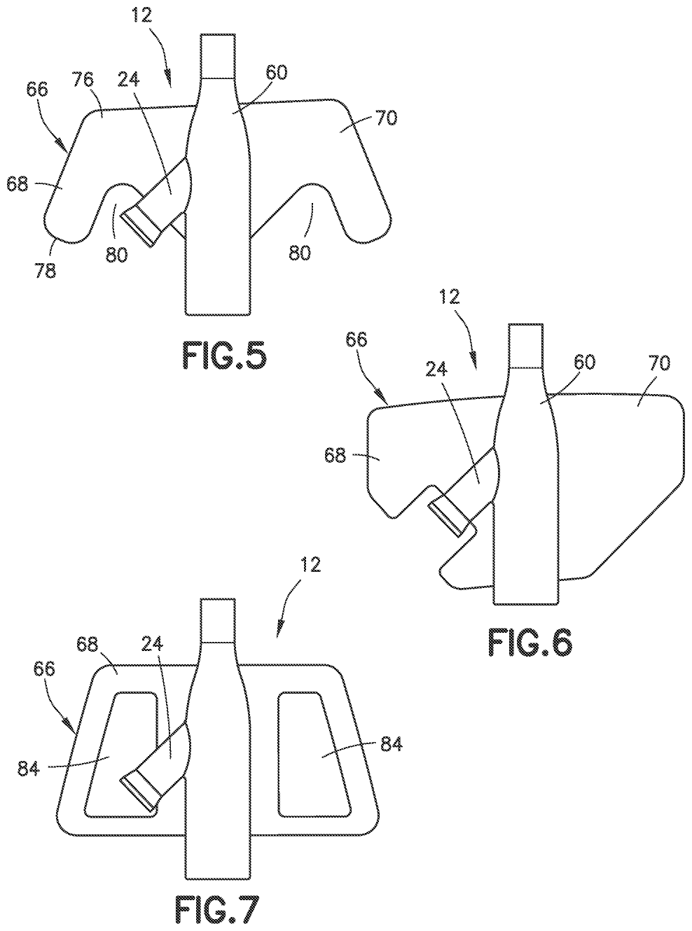

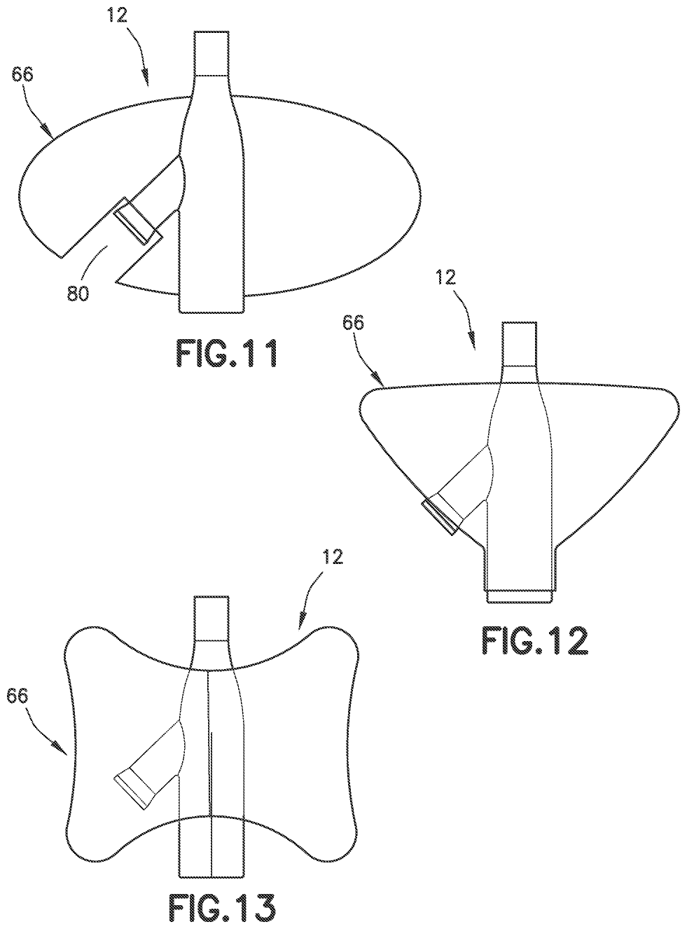

Referring to FIGS. 5, 6, 11, and 18, in certain aspects or embodiments, the first wing member 68 includes a first end 76 and a second end 78, with the first wing member 68 defining a recessed portion 80 adjacent to the inlet port 24 of the body 60. The recessed portion 80 of the first wing member 68 extends from the second end 78 of the first wing member 68 toward the first end 76 of the first wing member 68. The recessed portion 80 is configured to provide clearance around the inlet port 24. The stabilizer 66 may be symmetric, as shown in FIG. 5, with respect to the longitudinal axis L of the body 60, or may be asymmetric, as shown in FIGS. 6 and 11, with respect to the longitudinal axis L of the body 60.

Referring to FIG. 7, in one aspect or embodiment, the first and second wing members 68, 70 each define an opening 84 positioned entirely within a perimeter of the first and second wing members 68, 70.

Referring to FIG. 9, in one aspect or embodiment, the stabilizer 66 includes a first extension portion 86 extending from the first wing member 68 toward the second end 64 of the body 60 and a second extension portion 88 extending from the second wing member 70 toward the second end 64 of the body 60.

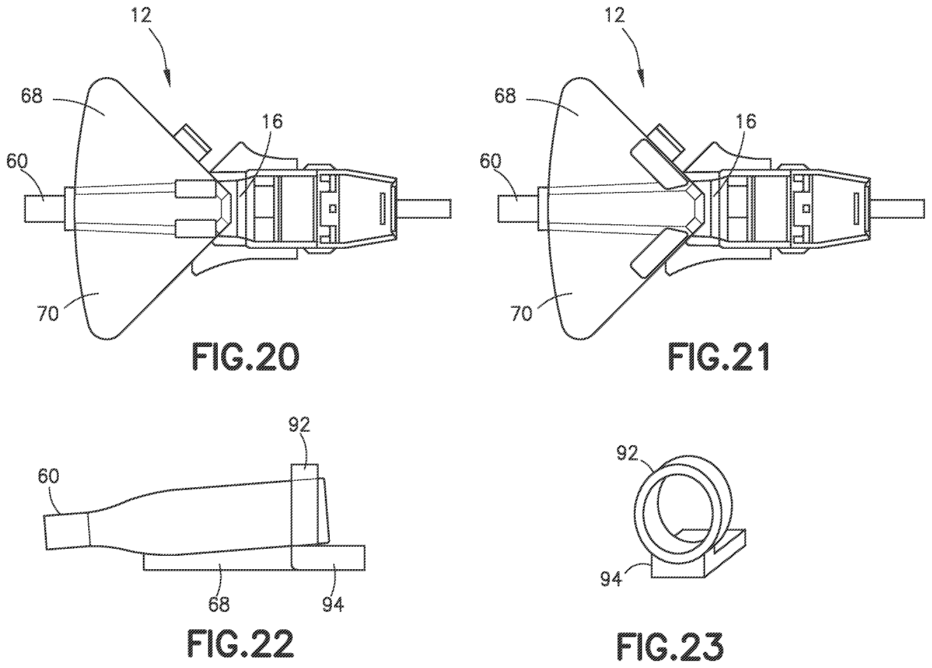

Referring to FIGS. 5-21, the stabilizer 66 may be symmetric or asymmetric with respect to the longitudinal axis L of the body 60. The stabilizer 66 may be triangle shaped, oval-shaped, or other various shapes. The stabilizer 66 may include various curves, cut-outs, and/or extensions. As shown more clearly in FIGS. 20 and 21, the stabilizer 66 is spaced from the needle hub assembly 16. In one aspect or embodiment, the stabilizer 66 is configured to provide clearance between the stabilizer 66 and the needle hub assembly 16 by extending the stabilizer 66 around the needle hub assembly 16.

Referring to FIGS. 22 and 23, in one aspect or embodiment, the stabilizer 66 includes a clip 92 configured to attach the stabilizer 66 to the body 60. The clip 92 is positioned on a base portion 94, with the base portion 94 forming a portion of the stabilizer 66. In one aspect or embodiment, the base portion 94 and the clip 92 are formed separately from the first and second wing members 68 70.

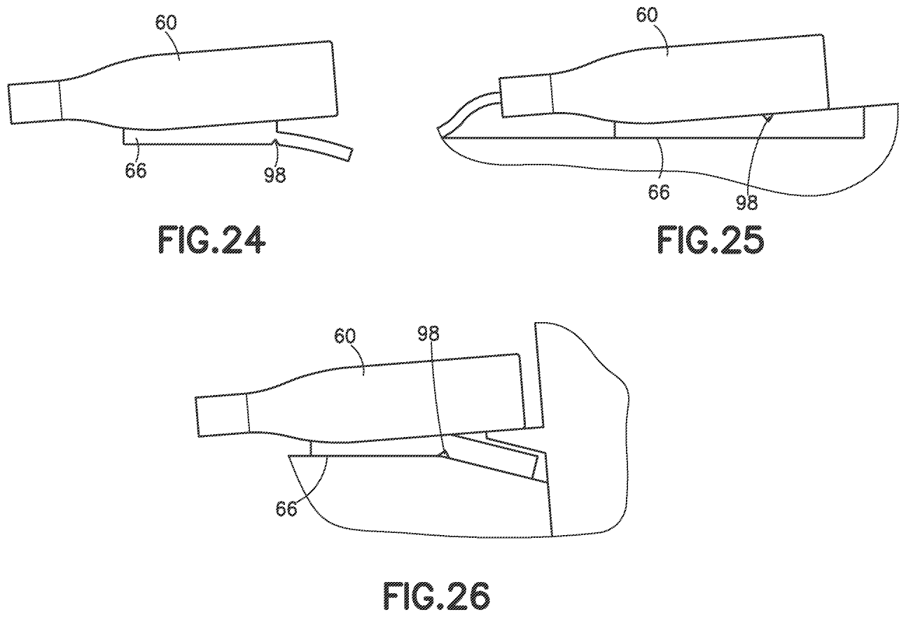

Referring to FIGS. 16, 19-21, and 24-26, in one aspect or embodiment, the portion of the stabilizer 66 extending beyond the inlet port 24 of the body 60 includes a hinged portion 98. In one aspect or embodiment, the hinged portion 98 is a living hinge. The living hinge may be formed via an undercut. Alternatively, as shown in FIG. 26, in one aspect or embodiment, the hinged portion 98 may be formed using two separate mold pulls. Rather than forming the hinged portion 98 with an undercut, the second mold pull flexes the hinged portion 98 out of the way.

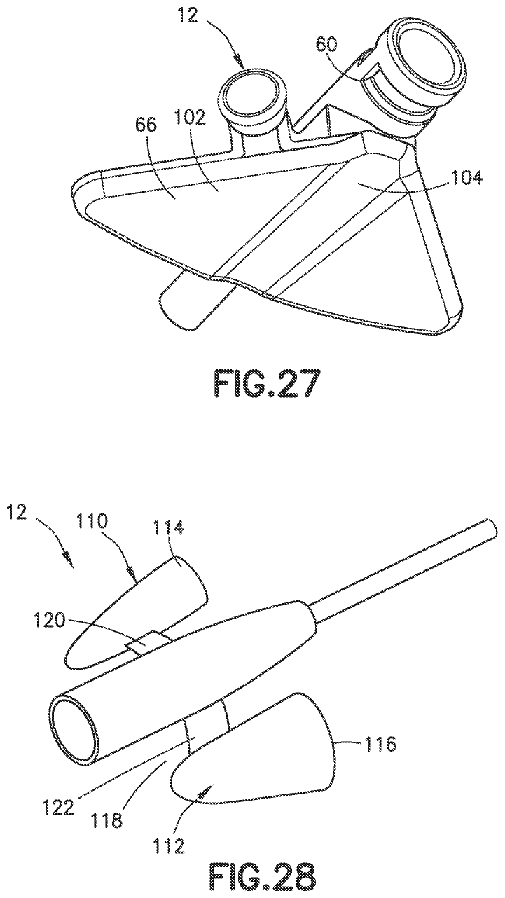

Referring to FIG. 27, in one aspect or embodiment, a bottom surface 102 of the stabilizer 66, which is configured to contact a skin surface of a patient, defines a recess 104 configured to align with a vein of a patient when the stabilizer 66 is in contact with a skin surface of a patient. A width and height of the recess 104 may be optimized to achieve a reduction of force. In one aspect or embodiment, the recess 104 is configured to decrease the likelihood of occluding the vein thereby increasing the likelihood of a successful blood draw.

Referring to FIG. 28, in one aspect or embodiment, rather than provide the first and second wing members 68, 70, the catheter adapter 12 includes a first support member 110 extending from the body 60 and a second support member 112 extending from the body 60, with the first and second support members 110, 112 configured to engage a skin surface of a patient and space the body 60 from the skin surface of the patient when the catheter 14 is inserted into the patient. The first and second support members 110, 112 each include a support surface 114, 116 spaced from a bottom surface 118 of the body 60 via a support arm 120, 122. The first support member 110 is positioned on an opposite side of the body 60 from the second support member 112. The support surface 114, 116 of each of the first and second support members 110, 112 may be formed from an elastomeric material. The first and second support members 110, 112 and the respective support arms 120, 122 may be formed from a plastic material, with the support surfaces 114, 116 formed onto the first and second support members 110, 112 via an overmolding process. The first and second support members 110, 112 are configured to provide stability to the catheter adapter 12 and prevent rocking of the catheter adapter 12 while also relieving pressure above the vein thereby reducing the chance of occluding the vein and leading to better blood draw outcomes.

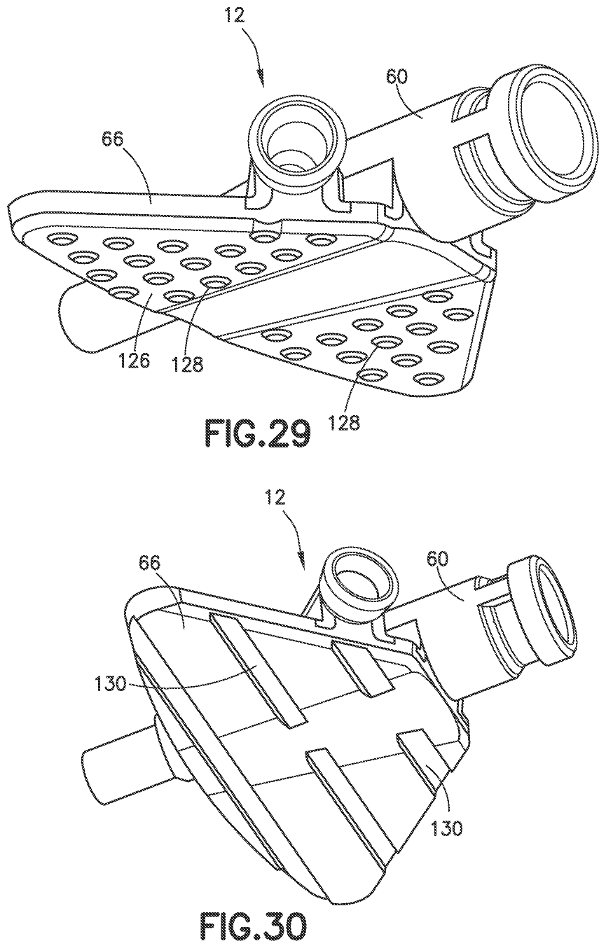

Referring to FIGS. 29 and 30, in certain aspects or embodiments, a bottom surface 126 of the stabilizer 66 includes a plurality of protrusions 128 and/or a plurality of channels 130. The plurality of protrusions 128 and the plurality of channels 130 are configured to reduce the force above a vein of a patient while also improving breathability of the skin directly underneath the stabilizer 66.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A catheter adapter comprising:
a body having a first end and a second end positioned opposite the first end, the body comprising an inlet port positioned between the first end and the second end, the body defining a longitudinal axis extending between the first end and the second end of the body;
a catheter configured to be inserted into a patient's vasculature, the catheter positioned at the first end of the body; and
a stabilizer comprising a first wing member and a second wing member, a portion of the stabilizer extending beyond the inlet port of the body in a direction extending along the longitudinal axis from the first end of the body to the second end of the body.

2. The catheter adapter of claim 1, wherein a bottom surface of the stabilizer is configured to contact a skin surface of a patient, and wherein the bottom surface of the stabilizer defines a plane forming an angle greater than zero relative to the longitudinal axis of the body.

3. The catheter adapter of claim 1, wherein the stabilizer is symmetric with respect to the longitudinal axis.

4. The catheter adapter of claim 1, wherein the stabilizer is asymmetric with respect to the longitudinal axis.

5. The catheter adapter of claim 1, wherein the first wing member includes a first end and a second end, the first wing member defining a recessed portion adjacent to the inlet port of the body, the recessed portion of the first wing member extending from the second end of the first wing member toward the first end of the first wing member.

6. The catheter adapter of claim 1, wherein the first and second wing members each define an opening positioned entirely within a perimeter of the first and second wing members.

7. The catheter adapter of claim 1, wherein the stabilizer comprises a first extension portion extending from the first wing member toward the second end of the body and a second extension portion extending from the second wing member toward the second end of the body.

8. The catheter adapter of claim 1, wherein the stabilizer comprises a clip configured to attach the stabilizer to the body.

9. The catheter adapter of claim 8, wherein the clip is positioned on a base portion, the base portion forming a portion of the stabilizer.

10. The catheter adapter of claim 1, wherein the portion of the stabilizer extending beyond the inlet port of the body comprises a hinged portion.

11. The catheter adapter of claim 10, wherein the hinged portion comprises a living hinge.

12. The catheter adapter of claim 1, wherein a bottom surface of the stabilizer is configured to contact a skin surface of a patient, and wherein the bottom surface of the stabilizer defines a recess configured to align with a vein of a patient when the stabilizer is in contact with a skin surface of a patient.

13. The catheter adapter of claim 1, wherein a bottom surface of the stabilizer is configured to contact a skin surface of a patient, and wherein the bottom surface of the stabilizer comprises a plurality of protrusions.

14. The catheter adapter of claim 1, wherein a bottom surface of the stabilizer is configured to contact a skin surface of a patient, and wherein the bottom surface of the stabilizer comprises a plurality of channels.

15. A vascular access device comprising:
a catheter adapter comprising:
a body having a first end and a second end positioned opposite the first end, the body comprising an inlet port positioned between the first end and the second end, the body defining a longitudinal axis extending between the first end and the second end of the body;
a catheter configured to be inserted into a patient's vasculature, the catheter positioned at the first end of the body;
a stabilizer comprising a first wing member and a second wing member, a portion of the stabilizer extending beyond the inlet port of the body in a direction extending along the longitudinal axis from the first end of the body to the second end of the body; and
a needle hub assembly connected to the body of the catheter adapter.

16. The vascular access device of claim 15, wherein the stabilizer is spaced from the needle hub assembly.

17. A catheter adapter comprising:
a body having a first end and a second end positioned opposite the first end, the body comprising an inlet port positioned between the first end and the second end, the body defining a longitudinal axis extending between the first end and the second end of the body;
a catheter configured to be inserted into a patient's vasculature, the catheter positioned at the first end of the body;
a first support member extending from the body; and
a second support member extending from the body, the first and second support members configured to engage a skin surface of a patient and space the body from the skin surface of the patient when the catheter is inserted into the patient.

18. The catheter adapter of claim 17, wherein the first and second support members each comprises a support surface spaced from a bottom surface of the body via a support arm.

19. The catheter adapter of claim 18, wherein the first support member is positioned on an opposite side of the body from the second support member.

20. The catheter adapter of claim 19, wherein the support surface of each of the first and second support members comprises an elastomeric material.
